(19) United States
(12) Patent Application Publication
(10) Pub. No.: US 2026/0283772 A1
(43) Pub. Date: Sep. 24, 2026

(54) INTRAURETHRAL DEVICE

(71) Applicant: INGENION MEDICAL LIMITED, London (GB)

(72) Inventors: Nabeel Jan, Middlesex (GB); Matthew Oliver Jones, Reading (GB); Edward Charles Cappabianca, Suffolk (GB)

(21) Appl. No.: 19/476,600

(22) PCT Filed: Apr. 17, 2024
(86) PCT No.: PCT/IB2024/000191

§ 371 Date: Oct. 17, 2025

Related U.S. Application Data
(60) Provisional application No. 63/460,167, filed on Apr. 18, 2023.

(51) Int. Cl. A61F 2/00; A61F 2/24; A61F 2/48

(52) U.S. Cl. CPC A61F 2/0022; A61F 2/2476; A61F 2/48; A61F 2210/009

(57) ABSTRACT

An intraurethral device comprising: a valve which is configured to be actuated between an open and a closed configuration by an external magnet; and a tube extending (31) from an outlet port of the valve, the tube (31) defining a passage for the flow of fluid, wherein at least a portion of the passage is an oval shape or an amy gdaloid shape.

### CROSS-REFERENCE TO RELATED APPLICATIONS

This application claims priority to, and the benefit of, U.S. Provisional Application No. 63/460,167, filed Apr. 18, 2023, the content of which is incorporated by reference herein in its entirety.

### TECHNICAL FIELD

Embodiments relate to intraurethral devices and, insertion systems for the placement of intraurethral devices, and the like.

### BACKGROUND

There is a desire, in general, to provide intraurethral devices, such as valves, which are capable of selectively controlling the flow of fluid therethrough. Such devices may be needed, for example, in relation to patients with urinary incontinence or retention deficiencies. This includes not only patients with a male urinary system but also patients with a female urinary system.

An example placement of such a valve in a male patient can be seen in FIG. 1, which is an extract of FIG. 3 of U.S. Pat. No. 6,066,088—reference numerals specifically referring only to prior art figures (FIGS. 1 and 2) in the present description will be presented in parentheses (reference numerals will be used (without parentheses in the description) to refer to FIGS. 3-15).

As can be seen, FIG. 1 shows a human male abdomen (30) in cross-section. A valve (1) may be located within a patient's bulbous urethra (26) generally at bulge (17) and generally upstream of the pendulous urethra (27)—in terms of the normal flow of fluid through the urethra. The valve (1) is coupled to a retention mechanism by a catheter (16) defining a lumen (21)—which as an outlet (25) at the valve (1) and an inlet defined by retaining loops (28) of the retention mechanism. The retention mechanism is located in the bladder (29) and includes the retaining loops (28) which hold the retention mechanism in place relative to the bladder neck (22).

The operation of the valve (1) between open and closed configurations is intended, therefore, to control the flow of fluid from the bladder (29) through the outlet (25) and to the pendulous urethra 27.

Important advances in this regard include the intraurethral magnetic valve of WO00/02499. FIG. 1 of this document is repeated as FIG. 2 of the present application for ease of reference. The document teaches an intraurethral magnetic valve (1) for insertion in the human urethra of a person suffering from incontinence. The intraurethral magnetic valve (1) includes a nonferromagnetic cylindrical housing (2), a valve seat assembly (36) attached to one end of the housing, a spherical magnetic valve element (14) disposed for universal movement in the housing, and a ring (10) for retaining the valve element which is attached at the other end of the housing. The valve seat assembly (36) has a nonferromagnetic valve seat (4) and ferromagnetic ring (5). The valve element (14) is maintained in closed check position against the valve seat (4) by attraction to the ferromagnetic ring (5), restricting the flow of fluid through the valve (1). An external magnet is used to impart magnetic torque and attraction to the valve element (14) displacing it from the valve seat (4) and opening the valve for fluid flow. On removal of the external magnet, the valve element (14) returns to its closed check position on the valve seat (4). The document also teaches a second embodiment in which the intraurethral magnetic valve (1) includes a mechanism having a spring for relieving excessive fluid pressure, in which the valve seat assembly (36), rather than being attached to the housing (2), is axially slidable within the housing against the force of the spring when excessive hydrostatic pressure is present to allow release of fluid through a passageway around the valve seat assembly (36).

Such advanced intraurethral magnetic valves seek to allow selective release of fluid from the bladder through use of an external magnet, in other words, a magnet external to the patient's body may be brought close to the internally fitted intraurethral magnetic valve to cause the actuation thereof from a closed configuration to an open configuration.

There are, however, problems with even these intraurethral magnetic valves. Some of these problems are particularly pertinent in patients with female urinary systems. Due to the shorter length of the urethra in such urinary systems, the valve may be located substantially at the urinary meatus (e.g. at the very end of the urethra). There is a greater risk, therefore, of migration of the valve towards the bladder. There is also an increased risk of infection with at least part of the valve extending out of the urethra or being located only just inside the urethra.

The placement of intraurethral valves has also been the subject of development. In particular, systems for the placement of intraurethral valves need to allow the valve, catheter, and an associated retaining mechanism to be pushed through the urethra into position. However, during placement in this manner, it is not uncommon for pulling and twisting of the valve, catheter, and their retaining mechanism to be required for accurate placement and navigation through the urethra. Typically, such placement systems require a portion which is manipulated by the practitioner (e.g. a surgeon or other medically qualified person) and which does not, therefore, enter the patient's urethra. All or part of this portion of the placement system needs to be removable from the valve, catheter, and their retaining mechanism after placement of the valve. Therefore, placement systems for intraurethral valve devices need to be able to navigate the valve, catheter, and their retaining mechanism into position through the urethra and need to be at least partially removable following such placement.

One such example is WO2008/067557 which teaches a system for implanting a catheter in a urethra. The system includes a catheter having a magnetic intraurethral valve disposed at one end portion of the catheter. The system also includes a tool having a tip member extending into an opening at the first end portion of the catheter to couple the tool to the catheter. The system also includes an element extending from the tool. The catheter has an internal path that provides a conduit for the element to be extended through the valve to engage a second end portion of the catheter. The tool is adapted to use the element to stress the catheter and stiffen the catheter along the length of the catheter Stiffening the catheter facilitates implantation of the catheter in the urethra by enabling pull forces, push forces, and torque forces.

Advancement in the area of intraurethral valve placement can also be found in WO2011032150. This document teaches an insertion facilitation device for facilitating insertion of a tube-like structure into a passage in a mammalian body. The device comprises: (a) an elongate stiffener including a proximal end portion and a distal end portion, the stiffener being adapted to extend in a lumen of the tube-like structure from a distal end portion thereof to a proximal end portion thereof, with the distal end portion of the stiffener at least partially within the distal end portion of the tube-like structure and the proximal end portion of the stiffener at least partially within the proximal end portion of the tube-like structure and provide a stiffness to the tube-like structure that is greater with the stiffener than without the stiffener, and be axially displaced within the tube-like structure relative to the tube-like structure; and (b) an engagement device that is adapted in an engaging condition thereof to releasably engage the stiffener and tube-like structure with the stiffener extending within the lumen of the tube-like structure such that either or both axial and radial displacement of the stiffener relative to the tube-like structure is substantially eliminated during insertion of the tube-like structure into a body passage, and in a disengaging condition thereof to release engagement of the stiffener and tube-like structure such that the stiffener is at least axially displaceable within the lumen of the tube-like structure and can be withdrawn from the tube-like structure at least so that the proximal end portion of the stiffener is in or adjacent the distal end portion of the tube-like structure.

These systems for the placement of intraurethral valves are, as can be seen from the aforementioned documents, relatively complex and there is a general desire to simply these systems.

Moreover, in a female urinary system, the valve is often located closer to or at the urinary meatus. Therefore, compared to placement in a male urinary system, the distance from the end of the valve closest to the urinary meatus to the meatus is much less and this means that placement tools can be specifically developed without having to design such tools for navigation through a significant length of the urethra (as is the case in many male urinary system valve systems).

These and other problems were considered in the significant advancements of WO2020089623. However, despite these advances there remains a continual need for further advancement and improved solutions to these and other problems relating to intraurethral devices, such as valve devices.

### SUMMARY

An aspect provides an intraurethral device including: a valve which is configured to be actuated between an open and a closed configuration by an external magnet; and a tube extending from an outlet port of the valve, the tube defining a passage for the flow of fluid, wherein at least a portion of the passage is an oval shape or an amygdaloid shape.

A device may further include a flange attached to the tube and configured to inhibit migration of the device.

The flange may have a generally circular outer shape.

The passage at a distal end thereof may be a circular shape.

The tube may extend over at least part of the valve.

The tube may include an external shape which narrows between the valve and a distal end of the passage.

The tube may be flexible.

Another aspect provides an intraurethral device including: a valve which is configured to be actuated between an open and a closed configuration by an external magnet; a catheter coupled in fluid communication with the valve such that actuation of the valve controls the flow of fluid through the catheter; a retention mechanism coupled to the catheter, wherein the retention mechanism is configured for deployment in a bladder; and an end piece including an attachment element, wherein the end piece is configured to be attached to a counterpart element of another part of the device which opposes the retention mechanism such that a length of the device is defined between the retention mechanism and the end piece, and wherein the attachment element is configured for removeable attachment to the other part of the device.

The attachment element and counterpart element may be correspondingly threaded elements configured to engage each other.

The attachment element and counterpart element may form a twist-lock attachment arrangement.

The attachment element may include a head portion configured to pass through an aperture defined by the counterpart element and the head portion may be configured to be rotated into abutment with an abutment portion of the counterpart element.

The counterpart element may include a frame which defines the aperture.

The frame may define a generally semi-circular aperture or a generally rectangular aperture.

The frame may include at least one chord beam.

The frame may include two chord beams.

The abutment portion and the head portion may include corresponding engagement features which, when engaged with each other, may be configured to inhibit rotation of the abutment portion and the head portion with respect to each other.

The engagement features may include at least one respective protrusion and recess.

The attachment element may be configured for axial rotation with respect to the counterpart element.

The attachment element and counterpart element may include a passage therethrough to permit the flow of fluid from the valve to through the end piece.

Another aspect provides an intraurethral device including: a valve which is configured to be actuated between an open and a closed configuration by an external magnet; and an counterpart element coupled to the valve, wherein the counterpart element is configured for removeable attachment to another part of the device or another element in a twist-lock attachment arrangement by engagement of the counterpart element with an attachment element of the other part or element.

The counterpart element may define an aperture configured to receive a head portion of the attachment element so that the head portion passes through the aperture and the counterpart element may have an abutment portion configured to abut the head portion following rotation of the head portion with respect to the counterpart element.

The counterpart element may include a frame which defines the aperture.

The frame may define a generally semi-circular aperture or a generally rectangular aperture.

The frame may include at least one chord beam.

The frame may include two chord beams.

The abutment portion and the head portion may include corresponding engagement features which, when engaged with each other, may be configured to inhibit rotation of the abutment portion and the head portion with respect to each other.

The engagement features may include at least one respective protrusion and recess.

The attachment element may be configured for axial rotation with respect to the counterpart element.

The attachment element and counterpart element may include a passage therethrough to permit the flow of fluid from the valve to through the end piece.

Another aspect provides an insertion system for an intraurethral device, the insertion system including: an insertion tube configured to receive, at least partially, the intraurethral device; and a plunger member configured to be at least partially received by the insertion tube such that movement of the plunger member through the insertion tube drives the intraurethral device from the insertion tube.

An insertion system may further include the intraurethral device and wherein the intraurethral device includes a valve which is configured to be actuated between an open and a closed configuration by an external magnet and a catheter coupled in fluid communication with the valve such that actuation of the valve controls the flow of fluid through the catheter, and optionally wherein the intraurethral device is a device as described above.

An intraurethral device may be used with the insertion system described above, the device including: a valve which is configured to be actuated between an open and a closed configuration by an external magnet; and an counterpart element coupled to the valve, wherein the counterpart element is configured for removeable attachment to another part of the device or another element in a twist-lock attachment arrangement by engagement of the counterpart element with an attachment element of the other part or element, wherein the plunger member includes the attachment element.

### BRIEF DESCRIPTION OF THE DRAWINGS

Features and advantages of the claimed subject matter will be apparent from the following detailed description of embodiments consistent therewith, which description should be considered with reference to the accompanying drawings.

FIG. 1 shows an extracted figure from WO00/02499

FIG. 2 shows an extracted figure from U.S. Pat. No. 6,066,088.

FIG. 3 shows an intraurethral device of the prior art.

FIGS. 4 and 5 show valves of the prior art.

FIGS. 6 and 7 show a version of a closure member.

FIGS. 8 and 9 show a version of a closure member.

FIGS. 10 and 11 show a version of a closure member.

FIG. 12 shows three versions of the device with different collapsible portions.

FIGS. 13 and 14 show a version of an end piece.

FIGS. 15 and 16 show a version of an end piece.

FIGS. 17 and 18 show a version of an end piece.

FIGS. 19, 20, and 21 show an insertion system of some versions.

FIGS. 22, 23, 24, and 25 show an attachment element counterpart element of some versions.

FIGS. 26, 27, 28, and 29 show an attachment element counterpart element of some versions.

FIGS. 30 and 31 show an attachment element counterpart element of some versions.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

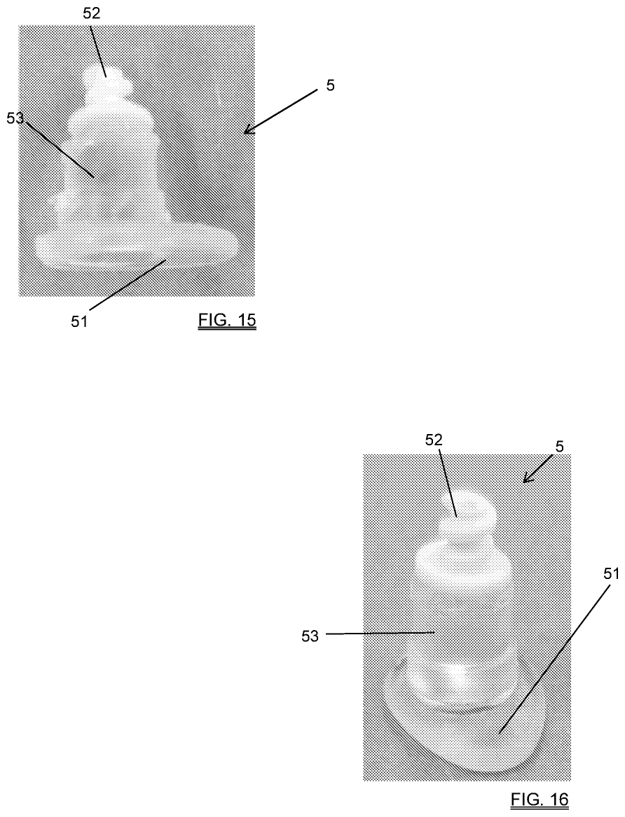

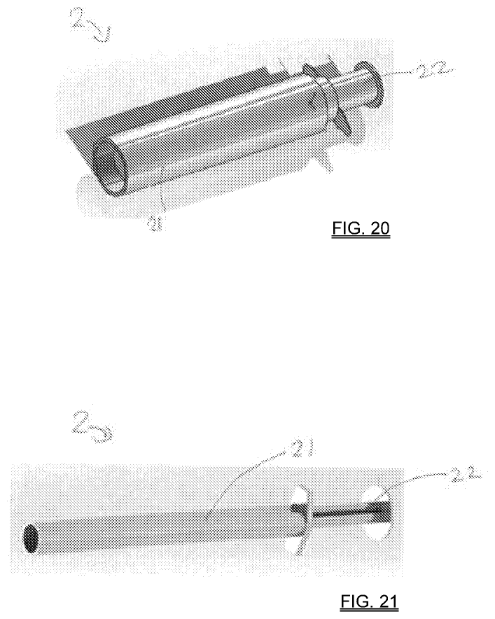

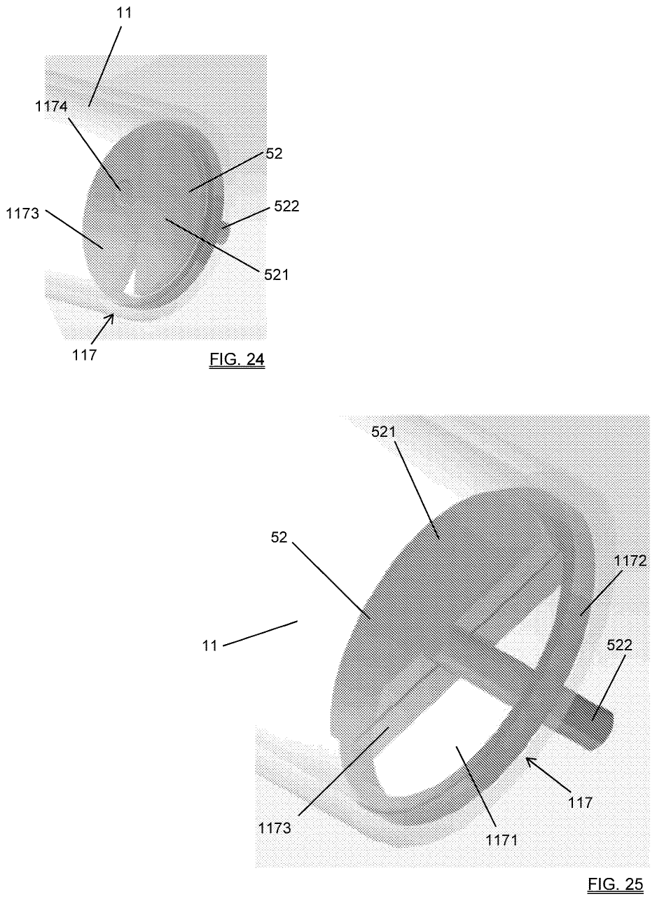

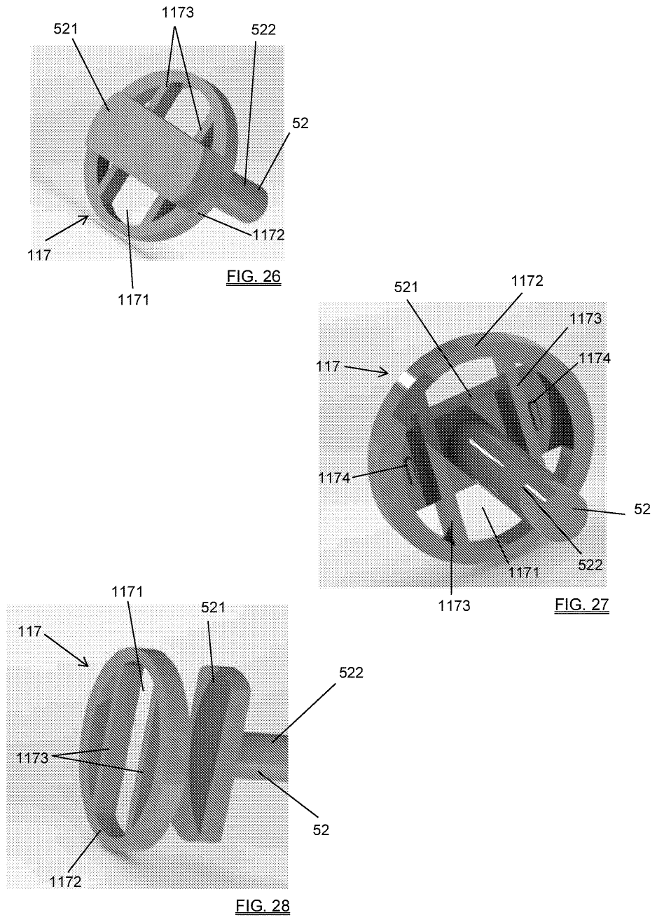

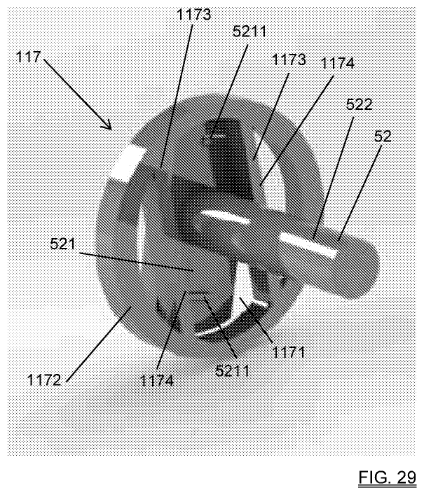

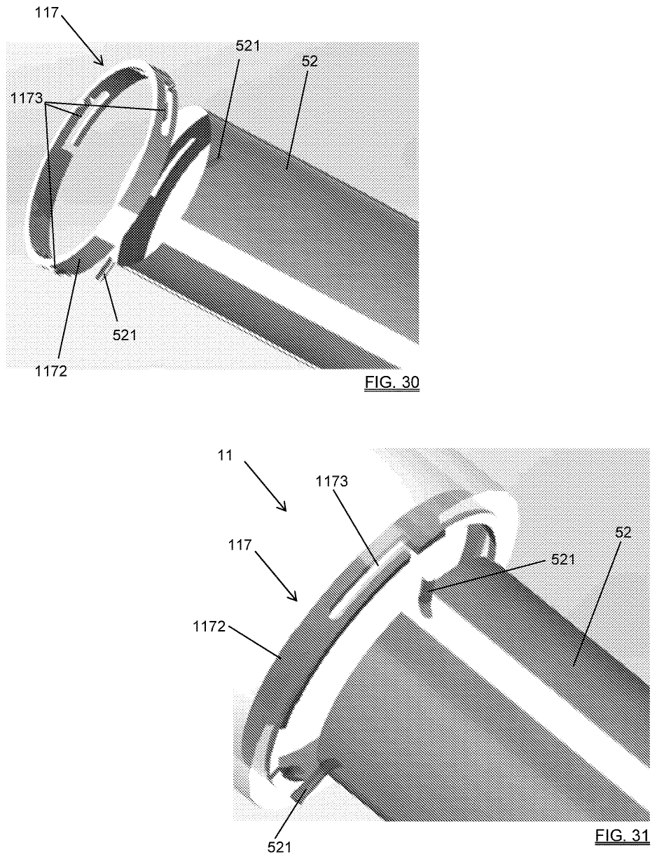

By way of overview, the present invention is generally directed to an intraurethral device comprising a unique valve. Various versions of the intraurethral device are shown and referred to in FIGS. 3 through 31 of the present application (specifically the device 1 including a valve 11). The valve 11 may be a magnetically actuated valve 11 for intraurethral placement. Therefore, the valve 11 may be an intraurethral magnetic valve 11.

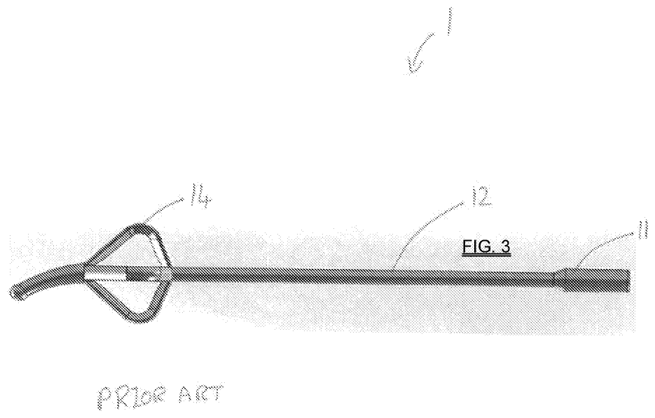

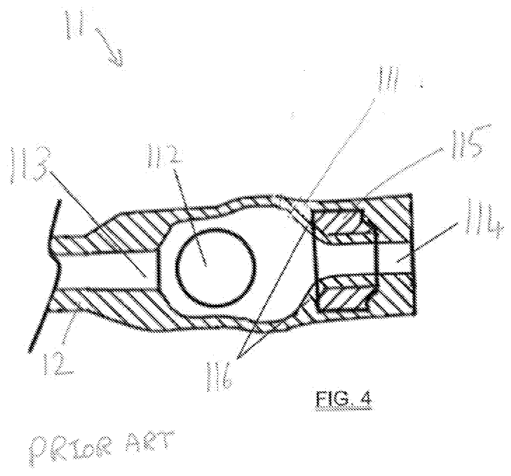

With reference to FIGS. 4 and 5 (which are depictions of prior art valves from WO2020/89623, the contents of which are incorporated by reference) by way of example, the valve 11 may include a valve main body 111 defining an internal valve volume in which is positioned a spherical magnetic valve element 112 (hereinafter “the valve element 112”). The internal valve volume has a length along a longitudinal axis of the valve main body 111. The longitudinal axis of the valve main body 111 extends from an inlet port 113 of the valve 11 to an outlet port 114 of the valve 11. The internal valve volume has a width along an axis perpendicular to the longitudinal axis of the valve main body 111.

The inlet port 113 and the outlet port 114 may both have a width (e.g. a diameter) which is less than that of the internal valve volume.

The internal valve volume is sized to receive the valve element 112, for example, as depicted in FIGS. 3 and 4. The internal valve volume may be sized to permit movement of the valve element 112 between a closed configuration and an open configuration—as described herein.

Towards the outlet port 114, there is a ferromagnetic member 115 which may be a ring. The ferromagnetic member 115 defines a passage through which fluid may pass. The ferromagnetic member 115 is depicted in the valve 11 in FIG. 3.

The ferromagnetic member 115 may be a continuous ring of ferromagnetic material. Accordingly, the ferromagnetic member 115 may be generally tubular in form. In some versions, the ferromagnetic member 115 may be ring-like, in that a complete ring may not be formed by ferromagnetic material which may form a ring-like structure formed of a plurality of ferromagnetic material segments.

The ferromagnetic member 115 may have a first end which is, when in the valve 11, towards the internal volume and a second end which is, when in the valve 11, away from the internal volume (relative to the first end). The first and second ends of the ferromagnetic member 115 may oppose each other across a length of the ferromagnetic member 115 (a length which may be parallel to the longitudinal axis of the valve 11 when the ferromagnetic member 115 is located in the valve 11).

The ferromagnetic member 115 may be coupled to the valve main body 111 and this may be achieved in several different manners.

In some versions, the valve main body 111 is formed from an elastomer material and may be formed from only the elastomer material. This elastomer material may be selected for its biocompatibility and/or the ability to impregnate the material with antimicrobial and/or antifungal agent. The ferromagnetic member 115 may be formed of a ferrous metal or ferrous metal alloy. Therefore, the valve body 111 may be formed from a relatively flexible material compared to the ferromagnetic member 115. In some versions, application of a force to the valve main body 111 may cause the valve main body 111 to deform elastically and for the internal valve volume width (in the direction of the force) to be reduced. In other words, the main body 111 may be resilient to crushing forces to which it may be exposed.

In some versions, the ferromagnetic member 115 may be embedded in at least part of the valve main body 111 with the valve main body 111 at least partially formed around the ferromagnetic member 115. In such versions, the formation of the valve main body 111 (or at least the part in which the ferromagnetic member 115 is embedded) may be a moulding process around the ferromagnetic member 115—which may, therefore, be placed inside a mould before elastomer material for the valve main body 111 (or part thereof) delivered to the mould (e.g. injected into the mould).

In some versions, the ferromagnetic member 115 may be secured by an interference fit within the valve main body 111. In such versions, the valve main body 111 may be formed separately from the ferromagnetic member 115, with a recess defined by the valve main body 111 for receipt of the ferromagnetic member 115. The ferromagnetic member 115 may then be inserted into the recess and held in place by, for example, an interference fit between the valve main body 111 and one or more surfaces of the ferromagnetic member 115. In some versions, a plug of material (which may be elastomer material such as that which may be used for the valve main body 111) may be used to secure, or help to secure the ferromagnetic member 115 in place. This plug may be located towards the outlet port 114 of the valve 11 and, indeed, may define the outlet port 114—the plug may be annular in form, for example, with the outlet port 114 defined by the plug.

In some versions, the ferromagnetic member 115 may be fitted to a spigot member of the valve main body 111. The spigot member may define a passage therethrough and the ferromagnetic member 115 may slide over the spigot member into position. A plug of material may then fit over the ferromagnetic member 115. The plug of material may be generally annular in form.

In some versions, adhesive may be used to secure, or help to secure, the ferromagnetic member 115 to the valve main body 111. In some versions, it will be understood that the spigot member may be part of the valve main body 111 and, likewise, the plug of material may form part of the valve main body 111.

In some versions, the valve main body 111 may be of a tubular form with one open end and with the ferromagnetic member 115 located at an opposing end (the opposing end forming the outlet which may be generally smaller in width than the open end). The open end may be configured to be fitted to another portion of the valve main body 111 and/or an end of a catheter 12—the other portion defining the inlet port 113 or an outlet of the catheter 12 forming the inlet port 113 for the valve 11. Therefore, the valve element 112 may be located within the valve main body 111 through the open end thereof prior to coupling to the other portion of the main body 111 and/or the end of the catheter 12 (the other portion of the main body 111 may be couplable to the catheter 12). The catheter 12 may be integrally formed with at least part of the valve 11 (such as the main body 111), or may be adhered thereto, or may be connected using a cohesive process (such as insert moulding).

In some versions in which the valve main body 111 is not moulded around the ferromagnetic member 115 during manufacture, the valve element 112 may be inserted through the outlet port 114 (or the end of the valve main body 111 at which the outlet port 114 will be located) prior to insertion of the ferromagnetic member 115 (and, for example, a plug). The elastomer material of the valve main body 111 may be configured to elastically deform to allow the insertion of the valve element 112 in this manner, for example.

The valve 11 includes a valve seat 116 which may be a non-ferromagnetic valve seat 116, for example. The valve seat 116 could be formed in a number of different manners according to versions. For example, the valve seat 116 may, in some versions, be formed by the material which forms the valve main body 111 in which the ferromagnetic member 115 is located.

The portion of the valve seat 116 which is configured to abut the valve element 112 may have a radius which matches or substantially matches an external radius of the valve element 112. In some versions, the portion of the valve seat 116 which is configured to abut the valve element 112 may be configured to deform elastically on contact with the valve element 112. Such arrangements may be configured to improve the seal between the valve seat 116 and the valve element 112.

In some versions, a separate ferromagnetic member 115 may not be used. For example, the valve seat 116 may be formed from an elastomer (e.g. silicone) including a filler material such as miniature steel or iron spheres or powder. In some versions the ferromagnetic member 115 is provided as a separate piece to the valve seat 116 but is, itself, formed from an elastomer (e.g. silicone) including a filler material such as miniature steel or iron spheres or powder.

In some versions, therefore, the end surface at the first end of the ferromagnetic member 115 may be covered by the material which forms the valve main body 111.

In some versions, the material of the valve main body 11 covers the end surface at the first end of the ferromagnetic ring 5; however, in this and some other versions, at least part of the passage through the valve 1 may be defined by the ferromagnetic ring 5 such that fluid passing therethrough may contact the ferromagnetic ring 5, for example (or at least part thereof). The valve seat 4 in these versions may be formed by the material covering the end surface at the first end of the ferromagnetic ring 5.

In some versions, with reference to FIG. 5 for example, the material of the valve main body 111 covers the end surface at the first end of the ferromagnetic member 115 but the rest of the ferromagnetic member 115 is also substantially covered by the material of the valve main body 111—such that fluid passing therethrough does not contact the ferromagnetic member 115 (or at least does not contact a substantial part thereof). The valve seat 116 in these versions may be formed by the material covering the end surface at the first end of the ferromagnetic member 115.

In some versions, a separate valve seat member is provided to form the valve seat 116. This separate valve seat member may be located adjacent the end surface at the first end of the ferromagnetic member 115 and may be secured to the ferromagnetic member 115 and/or at least partially embedded in the material of the valve main body 111. The valve seat member may be formed of a stiffer elastomer material than the valve main body 111, for example, or otherwise may have one or more properties which enhance the seal between the valve element 112 and the valve seat 116 (compared to using the material of the valve main body 111 for the valve seat 112). In some versions, the valve seat member may improve consistency in the formation of the valve seat 116 (again, compared to using the material of the valve main body 111 for the valve seat 112). The valve seat member may be formed from a different durometer silicone than the valve main body 111 (which may also be formed of silicone).

The device 1 (e.g. using the valve 11) may be actuatable between open and closed configurations (e.g. by the use of an external magnet, an external magnet being external to the device 1 and, when fitted, external to the body of the person to which the device 1 is fitted). In the open configuration, the valve element 112 is spaced apart from the valve seat 116 such that fluid may flow around the valve element 112 and through the outlet port 114. In the closed configuration, the valve element 112 is seated against the valve seat 116 such that the outlet port 114 is substantially sealed or sealed from fluid communication with the inlet port 113.

As will be appreciated, the valve element 112 may be biased toward the closed configuration by virtue of magnetic attraction to the ferromagnetic member 115. In addition, with the device 1 (e.g. the valve 11) located in a urethra as an intraurethral device (e.g. valve) and oriented with respect to the normal flow of fluid therethrough, the inlet port 113 may be upstream of the outlet port 114 with fluid, such that the flow of fluid through the valve 11 may also assist in biasing the valve member 112 towards the closed configuration.

Actuation of the valve 11 to the open configuration may be achieved through the use of a magnetic member. The magnetic member may be positioned external to the patient to which the valve 11 is fitted and moved into relative close proximity to the valve 11 to achieve actuation from the closed to the open configuration (through exposure of the valve 11 to the magnetic field of the magnetic member, wherein the magnetic field is sufficiently strong to achieve actuation).

Actuation to the closed configuration may be achieved by magnetic attraction between the valve element 112 and the ferromagnetic ring 115, and may be aided by one or both of gravity and the flow of fluid through the valve 11, for example.

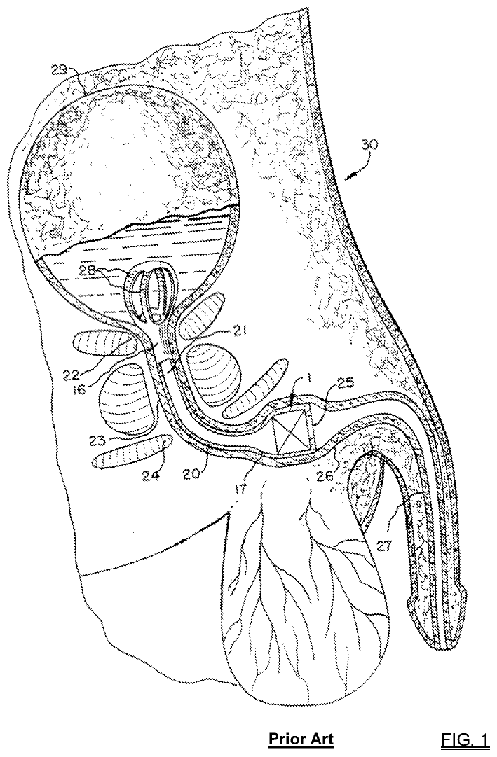

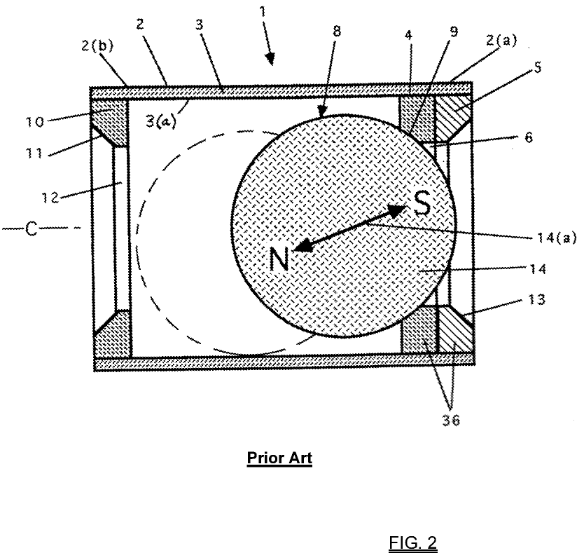

The valve 11 described herein may be fitted to a male patient or a female patient as an intraurethral magnetic valve 11. With reference to FIG. 1, the valve 11 described herein can take the place of the valve (1) shown in FIG. 1 and described in the document from which that figure was taken—see above for the document details.

As mentioned above, the valve 11 may be coupled to catheter 12. The catheter 12 provides and defines a fluid flow path to the valve 11 and, in particular, to the inlet port 113 of the valve 11. The valve 11 may be an intraurethral valve 11 and the catheter 12 may, accordingly, be coupled to a retention mechanism 14 which is configured to secure or help to secure the valve 11 and catheter 12 in the desired location. Some versions may not include a retention mechanism 14 and may be held in place by action of the catheter 12 and/or valve 11 against the urethra.

The retention mechanism 14 could take a number of different forms and is configured to be passed through a urethra to a desired location before being deployed. On deployment, the retention mechanism 14 helps to hold the catheter 12 and/or valve 11 in position. In some instances, deployment generally means the expansion or extension of at least part of the retention mechanism 14.

In some versions, the retention mechanism 14 may comprise a malecot—such as is shown in FIGS. 3 and 12. The retention mechanism 14 may include a plurality of arms or loops or rings which act, when deployed, to restrict movement of the catheter 12 and/or valve 11 which are coupled to the retention mechanism 14. The arms or loops or rings are, therefore, moveable from an undeployed or retracted configuration to a deployed or extended configuration. Herein the arms or loops or rings are examples of deployable members of the retention mechanism 14 and this term will be used hereinafter.

In some versions, therefore, there is provided a device 1 which includes: the valve 11, the catheter 12, and the retention mechanism 14.

The length of the catheter 12 from the valve 11 to the retention mechanism 14 may be selected based on the desired placement of the valve 11—as described herein, for example.

The retention mechanism 14 may, as described above, include a plurality of deployable members. These deployable members may extend radially outwardly with respect to the catheter 12 such that they define a part of the device 1 which is wider than the catheter 12 when deployed (but which may be retracted when in the undeployed configuration to be substantially the same width as the catheter 12, for example). The deployable members may define spaces therebetween. These spaces may provide fluid communication between a volume surrounding the retention mechanism and a lumen defined by the catheter 12. This volume, in the case of an intraurethral system, may be the bladder.

Accordingly, much as in FIG. 1, the retention mechanism 14 may locate in the bladder neck with the catheter 12 extending down (in terms of normal fluid flow direction) the urethra.

The valve 11 of some versions may be inserted or otherwise implanted using the systems disclosed in, for example, WO00/02499 and/or WO2011/032150 and/or WO2020/089623, the details of which are incorporated in their entirety by reference.

The valve 11 may be a valve as described in, for example, WO00/02499 and/or WO2011/032150 and/or WO2020/089623, the details of which are incorporated in their entirety by reference.

The magnetic member may be as described in, for example, WO00/02499 and/or WO2011/032150 and/or WO2020/089623, the details of which are incorporated in their entirety by reference.

In use, therefore, the device 1 may be located in a patient's bladder and urethra. In particular, the retention mechanism 14 may be located in the bladder, with the catheter 12 extending down the urethra, and the valve 11 located along the length of the catheter 12 or at/towards an end of the catheter 12 which is remote from the retention mechanism 14 (in some versions a length of the catheter 14 also extends from the outlet port 113 of the valve 11 such that the valve 11 is located between the two opposing ends of the catheter 12.

### Urinary Meatus Closure

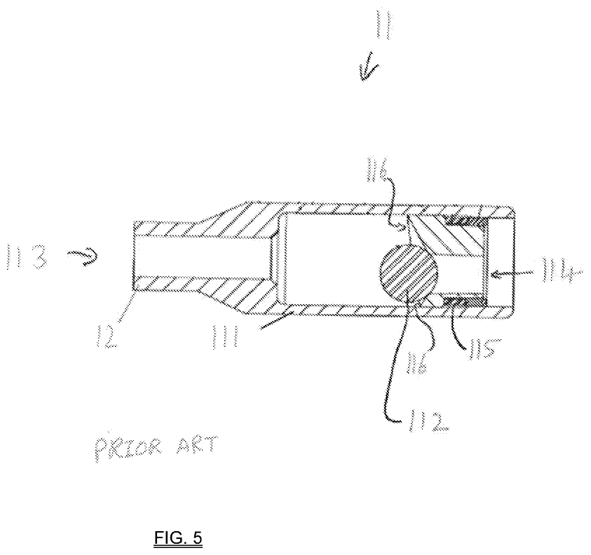

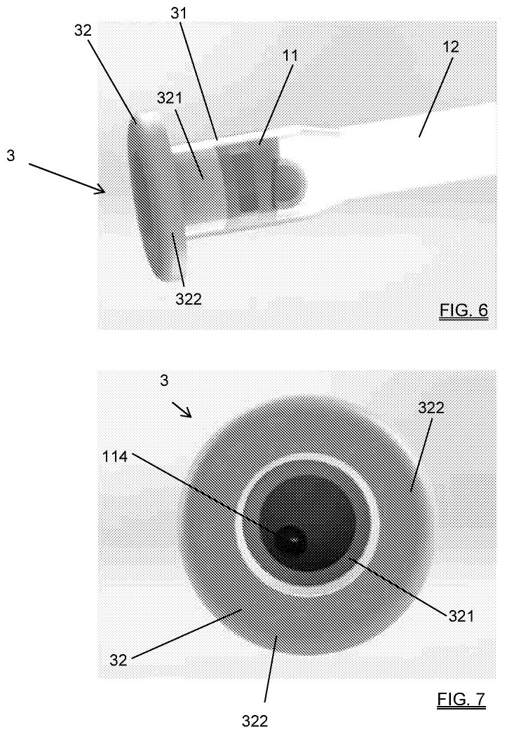

With reference to FIGS. 6-11, in some versions, the outlet port 114 of the valve 11 may be in fluid communication with a urinary meatus closure member 3 (which may be referred to herein as a “closure member 3”, for example).

The closure member 3 may include a tube 31 connected in fluid communication with the outlet port 114 and configured such that, with the retention mechanism 14 located in the bladder, a distal part of the tube 31 of the closure member 3 may extend to the urinary meatus and may be located outside of the urethra.

The distal part of the tube 31 (the proximal part being the part coupled to the valve 11, for example) may carry a flange 32. The flange 32 may have an external diameter which is larger than an external diameter of the tube 31. The flange 32 may also or alternatively have an external diameter which is larger than an external diameter of the valve 11. The flange 32 may have an external diameter which is sized so as to reduce the risk of migration of the flange 32 past the urinary meatus and into the urethra.

In some versions, the tube 31 may be formed from the same material as the valve main body 111 and/or the catheter 12. In some versions, the tube 31 is integrally formed with the valve 11. In some versions, the tube 31 extends over the valve 11 (e.g. over the valve main body 111) to sheath the valve 11 (or at least a part thereof). The tube 31 may be adhered or welded to the valve 11 for example.

In some versions, the flange 32 is integrally formed with the tube 31 (which may be integrally formed with the valve 11, as described herein). In some versions, the flange 32 is formed from the same material as the tube 31. In some versions, the flange 32 is formed from a different material from the tube 31. In some versions, the flange 32 is formed from a more rigid material than the tube 31 and this may help to prevent the flange 32 from folding and then passing into the urethra, for example. In some versions, the flange 32 is adhered or welded to the tube 31. In some versions the flange 32 comes in multiple parts—namely two annular parts, for example. The multiple parts of such versions of the flange 32 may be configured to receive a part of the tube 31 therebetween, such that joining of the two parts (e.g. through an adhesive, welding or and interference fit) may capture part of the tube 31 (e.g. the wall thereof around its circumference) to join the flange 32 to the tube 31. In some versions, one part of the flange 32 is an annular member or mushroom-shaped member defining an aperture and another part of the flange 32 is a tubular insert which is configured to fit within the annular member or mushroom-shaped member, with a part of the tube 31 sandwiched therebetween (an adhesive or welding may also be used).

In some versions, the tube 31 may have an internal diameter which defines a passage for the flow of urine therethrough (urine which passes through the valve 11 and into the tube 31).

The internal diameter may be substantially constant throughout a length of the tube 31 (see FIGS. 6 and 7, for example). An external diameter of the tube 31 may follow the internal diameter along the length of the tube 31. In some such versions, the external diameter may be generally the same as an external diameter of the valve 11 such that there is no substantive increase or decrease in the external diameter of the device 1 between the valve 11 and the tube 31. In some versions, the flange 32 may be provided by a mushroom-shaped member such that a neck portion 321 of the mushroom-shaped member is receivable within the tube 31 (e.g. may be shaped and sized to be so received, and so may have an external diameter which is generally the same as or marginally smaller than the internal diameter of the tube 31). The neck portion 321 may be, for example, adhered or welded to the tube 31 (e.g. within the tube 31).

The neck portion 321 may carry a flange portion 322 which extends beyond the tube 31 and which has a larger external diameter than the neck portion 321.

The tube 31 has an internal shape at its distal end (i.e. at the flange 32). This shape may be defined by the tube 31 itself or may be defined by a part of the flange 32 which may hold the tube 31 in such a manner so as to have that shape. In other versions, the flange 32 forms the distal end of the closure member 3. In any case, the tube 31 or flange 32 may form the distal end of the closure member 3 and either may define the internal shape of the passage therethrough (i.e. the passage through which urine will pass). This may be referred to herein as the passage's distal end shape, for example.

The passage also has a shape along a portion of its length between its distal end and the valve 11, and this may be referred to as an intermediate passage portion shape. This portion of the length may not include the distal end. The distal end of the passage may comprise a length of 2-30 mm (such that the intermediate passage portion may be located this far from the distal end of the tube 31 and/or the flange 32 and/or the closure member 3).

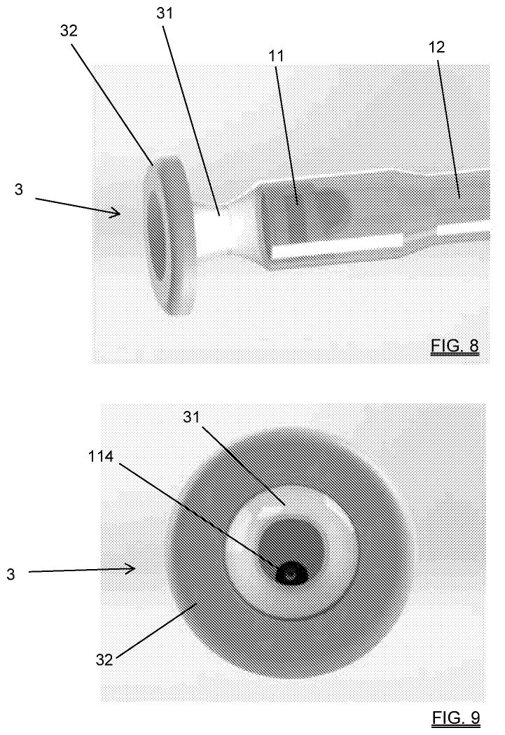

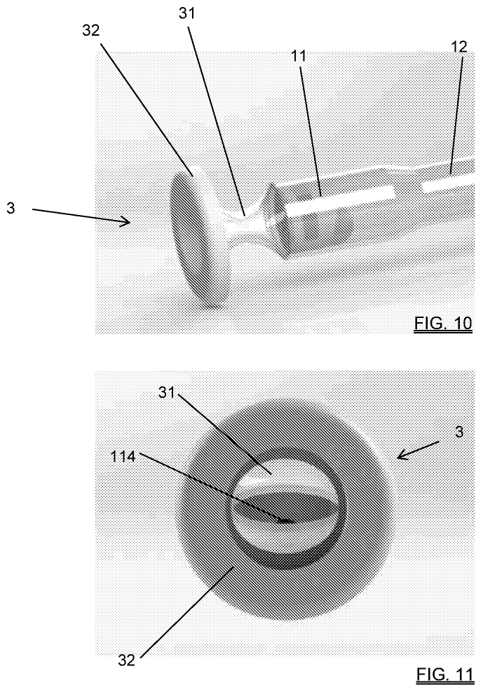

The passage's distal end shape may be a circular shape or may be oval shape or may be an amygdaloid shape (see FIGS. 10 and 11). The passage's intermediate portion shape may be a circular shape or may be an oval shape or may be an amygdaloid shape.

In some versions, the passage's shape in these portions is the shape through a cross-section of the closure member 3 which is perpendicular to its longitudinal axis (i.e. perpendicular to the direction of flow of urine in normal use).

In some versions, the passage's distal end shape extends (or is alternatively provided) along a length of the tube 31 towards the valve 11 and away from the flange 32.

In some versions, the passage's distal end shape is circular and the passage's intermediate portion shape may be an oval shape or may be an amygdaloid shape.

Accordingly, in some versions, at least a portion of the passage's shape may be an oval shape or may be an amygdaloid shape.

In some versions, an external shape of the tube 31 matches or otherwise corresponds with the internal shape of the tube 31.

In some versions, the cross-sectional size of the passage at or towards the distal end is smaller than that at the proximal end (i.e. adjacent the valve 11). In some versions, the passage is smallest in cross-section at a point between the flange 32 and the valve 11 (see FIGS. 8-11). In some versions, therefore, the tube 31 may narrow and then expand along the length between the valve 11 and the flange 32. This change in size may be a change in the internal cross-sectional size of the passage and/or the external cross-sectional size of the tube 31.

The passage at or towards the distal end of the tube 31 and/or flange 32 may be smaller than the outlet port 113 of the valve 11.

The smaller size of the passage at or towards the distal end of the tube 31 and/or flange 32 may help to reduce the risk of contaminants entering the passage and reaching the valve 11 and/or catheter 12, for example. In versions with a passage which is an amygdaloid shape, this may (with the tube 31 formed from an appropriately flexible material) at least partially open and close with the reduction or increase of an external (e.g. muscular) force. This may help urine control and/or avoiding contaminants. The pressure of urine flowing through the passage may help to open any closure of the passage in this area, for example.

The flange 32 is an example of an anti-migration member—i.e. a member which inhibits migration of the device into the bladder. Other forms of anti-migration member may be located at or towards the end of the device 1 which opposes the retention mechanism 4 (which inhibits movement of the device 1 out of the urethra through the urinary meatus).

### Size Adjustments

In the use of the device 1 in a female urinary system, the length of the urethra between the bladder and the urinary meatus is more important than in a male urinary system. This is because the retention mechanism 4 is located at the bladder and, in some versions, the anti-migration member is located at the meatus. If the device 1 is too long, then the device 1 may protrude excessively-causing discomfort. If the device 1 is too short, then the device 1 may not fit correctly or there may be discomfort from pressure applied by the retention mechanism 4 on the bladder.

In some versions, the catheter 12 may include a collapsible portion 4 (see FIG. 12) along at least part of its length. The collapsible portion 4 is configured to be collapsed to reduce the length of the device 1 or expanded to increase the length of the device 1. The collapsible portion 4 may be include one or more circumferential folds in a wall of the catheter 12 (e.g. forming a concertina portion).

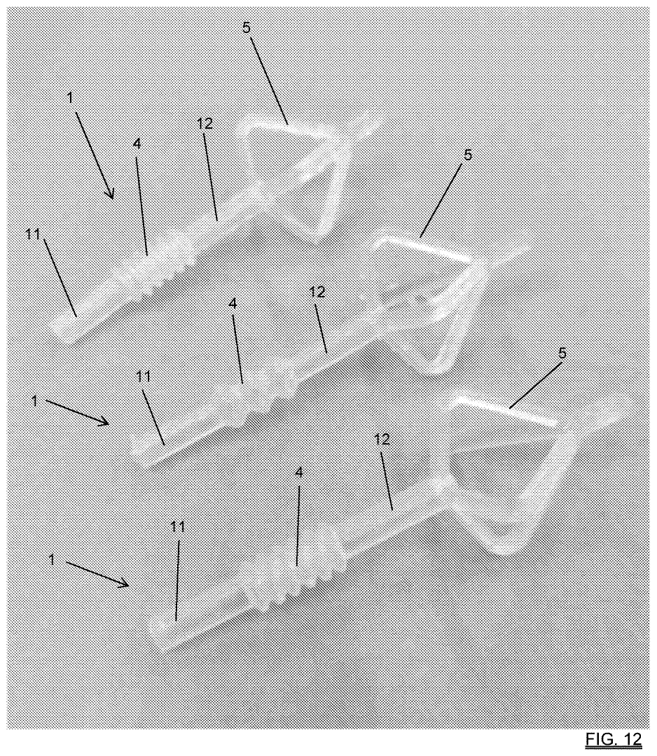

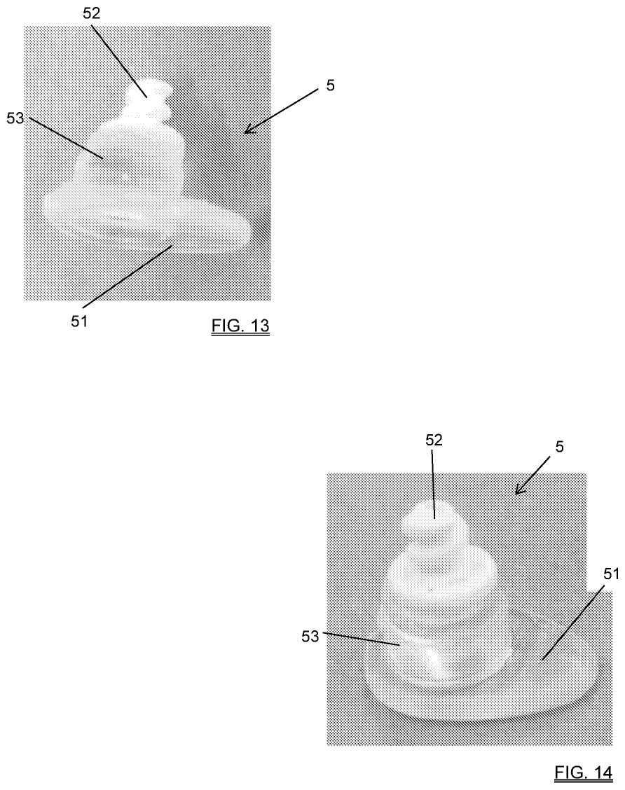

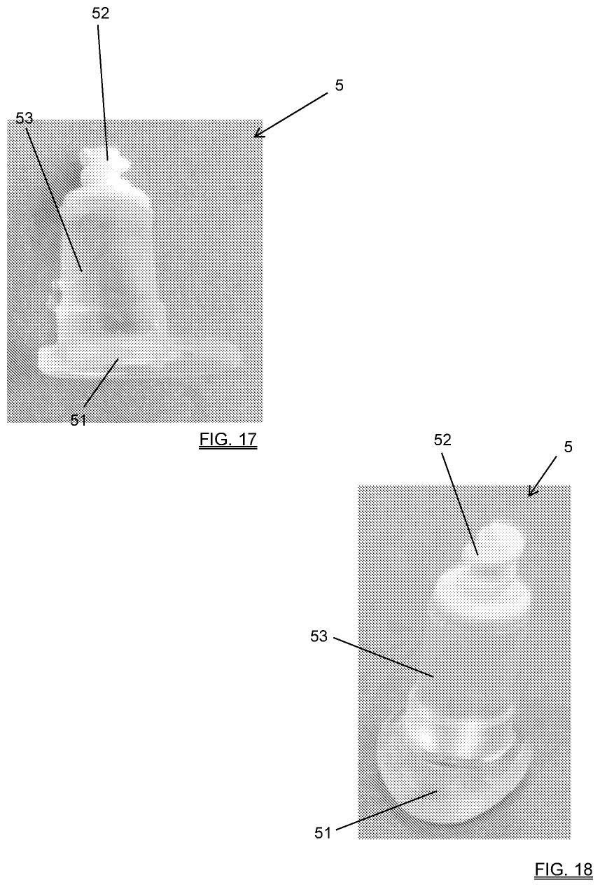

In some versions, there may be an end piece 5 (see FIGS. 13-18) which is configured to be fitted to an end of the device 1 which is located closest to the urinary meatus when the device 1 is fitted. This may be the valve 11 in some versions. The end piece 5 may be configured to be interchangeably fitted to that end of the device 1 such that one end piece 5 may be removed and a different one attached. In some versions, that end of the device 1 is configured to be coupled to any one of a plurality of different end pieces 5, wherein each end piece 5 may be a different size and/or shape. In the depicted examples, FIGS. 13 and 14 show the shortest depicted end piece and FIGS. 17 and 18 show the longest depicted end piece 5.

In some versions, the end of the device 1 which is located closest to the urinary meatus (e.g. the end of the valve 11 with the outlet port 114) may be provided with a threaded insert or a twist-lock (e.g. bayonet) fitting. In some versions, therefore, an end piece 5 (which may be one of many end pieces 5, which may be of a different size and/or shape) may include a corresponding threaded member or twist-lock (e.g. bayonet) fixing, such that the end piece 5 can be selectively attached to the end of the device 1. The end piece 5 may, therefore, include at attachment element 52 configured to engage a corresponding part of the device 1 of which it may be a part (e.g. the valve 11)—the threaded member or twist-lock fixing being examples).

An end piece 5 may include a flange 51 configured to act as an anti-migration member, which may be circular or oval or otherwise shaped to fit the body adjacent the urinary meatus. In some versions, the flange 51 may be configured to be cut to enable customisation of the shape for comfort (i.e. the flange 51 may be formed of a material which can be size manually using scissors or a knife). In some versions, the flange 51 may be moulded such that a custom mould may be created for a particular patient and flanges 51 made according to that mould.

The flange 51 of the end piece 5 defines an aperture through which urine will pass in use. The aperture is coupled to the attachment element 52 by a duct 53 which may be connected in fluid communication with the attachment element 52 and the aperture through the flange 51. Accordingly, the attachment element 52 may also define a passage which is in fluid communication with the duct 53 and aperture in the flange 51. In some versions, urine may pass through the valve 11, through the attachment element 52 of the end piece 5, through the duct 53, and out though the aperture defined by the flange 51 of the end piece 5.

The duct 53 may taper towards the attachment element 52.

In some versions, multiple different end pieces 5 may be provided which may have different lengths of duct 53, such that selection of different end pieces 5 enables different lengths of device 1 to be achieved. As such, the device 1 may be customised to fit a particular female urinary system.

In some instances, the end piece 5 could be configured to be replaced in-situ (i.e. with the device 1 in place within the patient). Regular changing of the end piece 5 (which may be for an identical end piece 5) would help to prevent contaminants causing infections or the like.

### Insertion

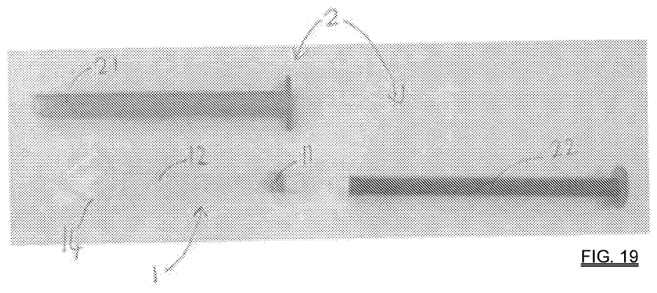

In some versions there is provided an insertion system 2 configured for use in inserting the device 1 into the urethra (see FIGS. 19-21, for example). The insertion system 2 may include an insertion tube 21 into which the device 1 is configured to be fitted such that the retention mechanism 14 is located within the insertion tube 21. In some versions, an end of the retention mechanism 14 may extend from an open end of the insertion tube 21 with the device 1 received by the insertion tube 21; however, the retention mechanism 14 may be in a collapsed state and may be held in that state by the insertion tube 21 (which may restrict the retention mechanism 14 from expanding).

Accordingly, the device 1 may be provided at least partially within the insertion tube 21 and the insertion tube may be inserted into a urethra. The insertion tube 21 may have a circular external cross-section and may be generally smooth, this may aid in the insertion of the tube 21 into the urethra (compared, for example, to attempting to insert the device 1 into the urethra). This may be particularly useful in examples in which there is provided a seal member is provided around at least part of the device 1.

The insertion system 2 may include a plunger member 22 which is configured to be at least partially received by the insertion tube 21. The plunger member 22 may include an engagement end which is configured to engage the device 1. Accordingly, the engagement end may include a portion which is configured to be at least partially received by the valve 11 or a part of the catheter 12. In some versions, the engagement end may include a portion which is configured to at least partially receive the valve 11 or a part of the catheter 12. The engagement end may engage the device 1 such that movement of the plunger member 22 causes movement of the device 1. Accordingly, with the device 1 at least partially received by the insertion tube 21, the plunger member 22 may engage the device 1 (with a part of the plunger member 22 extending from one end of the insertion tube 21) and movement of the plunger member 22 though the insertion tube 21 may push the device 1 from the opposing end of the insertion tube 21. As such, the end of the plunger member 22 (which opposes the engagement end) may include a plate configured to aid a user in manually pushing the plunger member 22. In some versions, the insertion tube 21 may include one or more (e.g. two) finger protrusions, such that a user can use the fingers of one hand to hold the insertion tube 21 and press the plunger member 22 (e.g. with their thumb), in much the same manner as a syringe is operated.

This insertion system 2 may provide a relatively simple system for insertion of a device 1. The insertion system 2 may be pre-lubricated and lubrication may include lubrication of both the inner and outer surfaces of the insertion tube 21 or just one of these surfaces.

The insertion tube 21 may be a rigid member or may be more rigid than the device 1. The insertion tube 21 may, therefore, aid in the insertion of the device 1.

As will be appreciated, the deployment of the device 1 from the insertion system 2 involves the pushing of the device 1 from the insertion tube 21 (using the plunger member 22). As the retention mechanism 14 leaves the confines of the insertion tube 21, the retention mechanism 14 may expand or deploy (e.g. in the bladder). The plunger member 22 may then be disengaged from the device 1 and then removed from the urethra—leaving the device 1 retained in the urethra (and bladder).

The engagement between the engagement end and the device 1 could take a number of different forms, including a threaded or twist-fit (e.g. bayonet) engagement. In some versions simple abutment is all that is provided between the engagement end and the device 1. In some versions, the engagement between the engagement end and the device 1 uses the same part of the device which is used for connecting an end piece 5 as described herein. Accordingly, in some versions, the engagement end may include an attachment element 52 as described herein.

### Attachment Elements

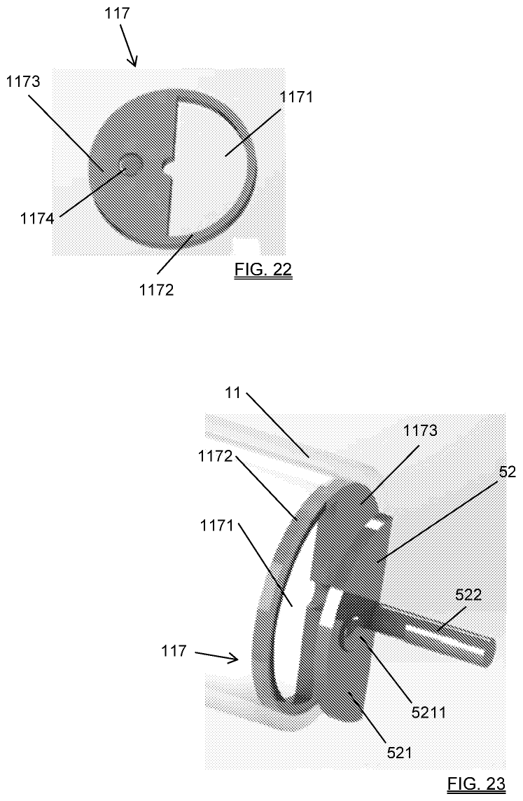

With reference to FIGS. 22-32 but also FIGS. 12-18, the use of attachment elements 52 has been described in relation to both the end piece 5 and the plunger member 22, for example. Various different attachment elements 52 are possible. For example, the attachment element 52 may include a male threaded part which is configured to mate with a female threaded part of the valve 11 (or some other part of the device 1)—see FIGS. 12-18. The female threaded part may be an insert which is embedded (e.g. over moulded) during formation of the valve 11, for example.

As mentioned herein, twist-fit engagement is also possible in some versions (e.g. a bayonet fitting).

Some example twist-fit engagement attachment elements 52 and their counterpart elements 117 of the device 1 (e.g. of the valve 11) are now described with reference to FIGS. 22-32.

In some versions, the attachment element 52 is configured to be pushed past a position of the counterpart element 117 and then rotated with respect thereto to lock the attachment element 52 to the counterpart element 117. Once locked together, axial movement of the attachment element 52 in at least one direction (and in some versions in both axial directions) will cause a corresponding movement of the counterpart element 117.

In some versions, the counterpart element 117 may define an aperture 1171 which is configured to receive a head portion 521 therethrough. In some versions, the counterpart element 117 includes a frame 1172 which defines, at least partially if not completely, the aperture 1171 through the counterpart element 117. The counterpart element 117 may also include an abutment portion 1173. The abutment portion 1173 may be a part of the frame 1172 or may be coupled to the frame 1172 or some other part of the valve 11, in some versions.

The attachment element 52 head portion 521 may be configured to be received by the aperture 1171 through the counterpart element 117 and then axially rotated (with respect to the counterpart element 117) such that the head portion 521 (formerly aligned with the aperture 1171) becomes aligned with the abutment portion 1173 such that the head portion 521 and abutment portion 1173 abut to inhibit or prevent removal of the attachment element 52 from the counterpart element 117. In such a configuration movement of the attachment element 52 axially will, through the abutment of the head portion 521 and the abutment portion 1173, cause corresponding axial movement of the counterpart element 117 (in the same direction). Accordingly, in such a configuration, the attachment element 52 and counterpart element 117 are coupled (and so my be generally said to be couplable).

Axial rotation of the attachment element 52 with respect to the counterpart element 117 to realign the head portion 521 with the aperture 1171 of the counterpart element 117 may then allow the head portion 521 to pass through the aperture 1171, such that the attachment element 52 can be decoupled. This may allow removal of one or more elements from the device which were coupled through the use of the attachment element 52 and counterpart element 117 to the device 1 and may include removal of part of the device 1 from another part of the device 1, for example.

The attachment element 52 head portion 521 and the counterpart element 117 could take a number of different forms.

In some examples (see FIGS. 22-25, for example), the attachment element 52 includes a shaft 522 and the head portion 521. The head portion 521 may be in the form of a member extending radially from the shaft 522. In some versions, the head portion 521 is semi-circular (e.g. with the centre of the circle aligned with a central longitudinal axis of the shaft 522.

The counterpart element 117 may include the frame 1172 in the form of an annular ring with an internal diameter which is equal to or marginally larger than an external diameter of the head portion 521 such that the head portion 521 can pass through the annular ring. The counterpart element 117 may include the abutment portion 1173 and this may be generally semi-circular and may extend across approximately half of the opening defined by the frame 1172. The aperture 1171 may, therefore, be defined by the frame 1172 and the abutment portion 1173 such that the aperture 1171 is generally semi-circular. The abutment portion 1173 may include a slot therethrough which is configured to receive at least part of the shaft 522, the slot may be semi-circular in shape. The slot may be generally locate at the centre of the opening of the frame 1172.

As such, in some such versions, the attachment element 52 may be presented to the counterpart element 117 with the head portion 521 and aperture 1171 generally aligned. The head portion 521 may be passed through the aperture 1171 (with the shaft 522 at least partially received by the slot in the abutment portion 1173). The attachment element 52 may then be rotated with respect to the counterpart element 117 (e.g. axially) such that the head portion 521 moves to abut the abutment portion 1173 such that, in this position, the head portion 521 is inhibited or prevented from passing back through the aperture 1171.

In some versions, the abutment portion 1173 and the head portion 521 are each in the form of respective parts of discs (i.e. having flat opposing sides).

In some versions, the head portion 521 is provided with a hinge which couples the head portion 521 to the shaft 522 such that the head portion 521 can rotate between a position (as depicted) in which it extends perpendicular to the shaft 522 to a position (not depicted) in which it is folded to be adjacent the shaft. This may aid insertion, for example.

In some versions, the abutment portion 1172 and the head portion 521 include respective engagement features 1174,5211. The engagement features 1174,5211 are configured to engage each other to inhibit rotation of the abutment portion 1172 and the head portion 521 with respect to each other when the two portions 1172, 521 abut each other. In some versions, the engagement features 1174,5211 include a corresponding protrusion and recess which interlock with each other, for example. In some versions, the protrusion may be generally circular and the recess may be generally circular. The engagement features 1174,5211 may be provided spaced apart from the rotational axis of the attachment element 52 during use.

The attachment element 52 and/or the counterpart element 52 may be formed from a plastics material or steel (e.g. stainless steel).

As will be appreciated, this abutment of the abutment portion 1172 and the head portion 521 may inhibit or prevent axial movement in one direction. Axial movement in the other direction may be inhibited or substantially prevented by abutment of the head portion 521 against a part of the valve 11 adjacent the abutment portion 1172 (so that a channel for the head portion 521 is defined between the abutment portion 1172 and the part of the valve 11). Axial movement in the other direction may be inhibited or substantially prevented by abutment of an element to which the attachment element 52 is attached or forms a part and the valve 11 or another part of the device 1 (such an element may be the end piece 5, for example, or the closure member 3, for example, or the plunger member 22, for example).

As will be appreciated, the roles of the attachment element 52 and the counterpart element 117 may be reversed (e.g. so that the valve 11 includes or is attached to the counterpart element 117 and the attachment element 52 is part of or is attached to another element (such as the end piece 5, closure member 3, or plunger member 22, for example).

In some versions, the head portion 521 may have a different shape to some of the versions described above (see FIGS. 26 to 29, for example). For example, the head portion 521 may be generally rectangular in shape (the short sides of which may be curved). The head portion 521 may be symmetrical about the rotational axis in use (i.e. about the longitudinal axis of the shaft 522).

In some versions, the frame 1172 may be in the form of an annular member with two chord beams located on either side of a centre of the annular member and extending across the opening defined by the annular member. As such, the two chord beams may define a gap therebetween (along with parts of the annular member) in which the centre of the opening is located and this gap may provide the aperture 1171 (e.g. this gap may be the aperture 1171). The head portion 521 may be correspondingly shaped to this gap (i.e. taking a circle of corresponding size to the opening defined by the annular member two chords of that circle may be two opposing edges of the head portion 521, with two other opposing edges being parts of the circumference of that circle). The shaft 522 may extend from a central location with respect to the head portion 521, as will be appreciated.

The head portion 521 may, therefore, be passed through the aperture 1171 and then rotated in the same manner as described above. The head portion 521 may then abut the chord beams which, in such versions, therefore comprise the abutment portion 1173.

In some versions, the engagement features 1174, 5211 may include one or more protrusions 5211 which extend from respective parts of the head portion 521 and which abut respective edges of the or each chord beam. Accordingly, in such versions, the chord beam(s) may also form the engagement feature(s) and, in particular, edge(s) thereof may form the engagement feature(s).

In some versions, there may be only one chord beam.

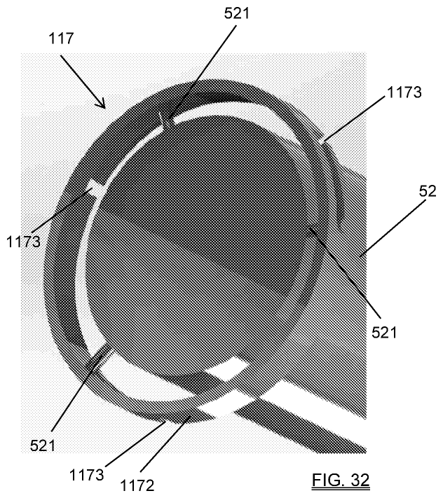

In some versions, the attachment element 52 may take a different form (see FIGS. 30-32, for example) and may include two or more protrusions (e.g. protrusions which extend radially from an element to which the attachment element 52 is attached or of which it forms a part, or from a tubular body section of the attachment element 52). These protrusions may be cylindrical. These protrusions may each form the head portion 5 and there may, therefore, be a plurality of head portions 521 in some versions.

The counterpart element 52 in some such versions includes the frame 1172 in the form of an annular member which defines L-shaped slots in its circumference. These L-shaped slots may each include an opening to receive a respective protrusion of the attachment element 52. These L-shaped slots may define respective channels from their openings which pass through a bend (e.g. a 90 degree bend) such that axial movement of the attachment element 52 with respect to the counterpart element 52 causes receipt of the protrusions of the attachment element 52 through the openings defined by the counterpart element 52. The attachment element 52 may then be axially rotated with respect to the counterpart element 52 such that the protrusions travel along their respective channels in the counterpart element 52 and around the bend. In some such versions, the parts of the frame 1172 defining the L-shaped slots form the abutment portion 1173.

For the sake of ease of reference, device 1 versions have been described with reference to the passage and flow of urine. As will be appreciated, there may be other fluids which pass through the device 1 and it is appropriate, therefore, to refer to the flow and passage of fluids in general.

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1.-7. (canceled)

8. An intraurethral device comprising:
a valve which is configured to be actuated between an open and a closed configuration by an external magnet;
a catheter coupled in fluid communication with the valve such that actuation of the valve controls the flow of fluid through the catheter;
a retention mechanism coupled to the catheter, wherein the retention mechanism is configured for deployment in a bladder; and
an end piece comprising an attachment element, wherein the end piece is configured to be attached to a counterpart element of another part of the device which opposes the retention mechanism such that a length of the device is defined between the retention mechanism and the end piece, and wherein the attachment element is configured for removeable attachment to the other part of the device.

9. The device of claim 8, wherein the attachment element and counterpart element are correspondingly threaded elements configured to engage each other.

10. The device of claim 8, wherein the attachment element and counterpart element form a twist-lock attachment arrangement.

11. The device of claim 10, wherein the attachment element includes a head portion configured to pass through an aperture defined by the counterpart element and the head portion is configured to be rotated into abutment with an abutment portion of the counterpart element.

12. The device of claim 11, wherein the counterpart element includes a frame which defines the aperture.

13. The device of claim 12, wherein the frame defines a generally semi-circular aperture or a generally rectangular aperture.

14. The device of claim 13, wherein the frame includes at least one chord beam.

15. The device of claim 14, wherein the frame includes two chord beams.

16. The device of claim 11, wherein the abutment portion and the head portion include corresponding engagement features which, when engaged with each other, are configured to inhibit rotation of the abutment portion and the head portion with respect to each other.

17. The device of claim 16, wherein the engagement features include at least one respective protrusion and recess.

18.-19. (canceled)

20. An intraurethral device comprising:
a valve which is configured to be actuated between an open and a closed configuration by an external magnet; and
an counterpart element coupled to the valve, wherein the counterpart element is configured for removeable attachment to another part of the device or another element in a twist-lock attachment arrangement by engagement of the counterpart element with an attachment element of the other part or element.

21. The device of claim 20, wherein the counterpart element defines an aperture configured to receive a head portion of the attachment element so that the head portion passes through the aperture and the counterpart element has an abutment portion configured to abut the head portion following rotation of the head portion with respect to the counterpart element.

22. The device of claim 20, wherein the counterpart element includes a frame which defines the aperture.

23. The device of claim 22, wherein the frame defines a generally semi-circular aperture or a generally rectangular aperture.

24. The device of claim 23, wherein the frame includes at least one chord beam.

25. The device of claim 24, wherein the frame includes two chord beams.

26. The device of claim 20, wherein the abutment portion and the head portion include corresponding engagement features which, when engaged with each other, are configured to inhibit rotation of the abutment portion and the head portion with respect to each other.

27. The device of claim 26, wherein the engagement features include at least one respective protrusion and recess.

28. The device of claim 20, wherein the attachment element is configured for axial rotation with respect to the counterpart element.

29.-31. (canceled)

32. An intraurethral device and insertion system combination,
the insertion system comprising:
an insertion tube configured to receive, at least partially, the intraurethral device; and
a plunger member configured to be at least partially received by the insertion tube such that movement of the plunger member through the insertion tube drives the intraurethral device from the insertion tube; and
the intraurethral device comprising:
a valve which is configured to be actuated between an open and a closed configuration by an external magnet; and
a counterpart element coupled to the valve, wherein the counterpart element is configured for removeable attachment to another part of the device or another element in a twist-lock attachment arrangement by engagement of the counterpart element with an attachment element of the other part or element, wherein the plunger member includes the attachment element.
